(19) United States
(12) Patent Application Publication
(10) Pub. No.: US 2008/0134403 A1
(43) Pub. Date: Jun. 12, 2008

(54) Hip Protector

(76) Inventor: Julian Minns, Burnley (GB)

Correspondence Address: Thomas E. Sisson, 112 E. Pocan Street, Suite 2400, San Antonio, TX, 78205, US

(21) Appl. No.: 11/631,134

(22) PCT Filed: Jul. 1, 2005
(86) PCT No.: PCT/GB05/02567

§ 371 Date: Jul. 20, 2007

(30) Foreign Application Priority Data
Jul. 1, 2004 (GB) 0414734.4

(51) Int. Cl. A41D 13/00; A61F 13/00

(52) U.S. Cl. 2/22; 602/54

(57) ABSTRACT

A hip protector comprises a padded region and an adhesive region surrounding the padded region, and a template, the template comprising a first element for alignment with an anterior superior iliac crest, a second element for alignment with a substantially vertical axis, and a third element which is substantially aligned with the greater trochanter when the first and second elements are aligned respectively with the anterior superior iliac crest and the vertical axis.

### FIELD OF THE INVENTION

This invention relates to hip protectors, and in particular to a hip protector of the type that is attached to the skin covering the hip bone.

### BACKGROUND OF THE INVENTION

In recent years it has become common to use padded devices to protect the hip. These devices are used for two different purposes. First, they are used to protect the hip from damage in the event of a fall, and second they are used to reduce the risk of pressure sores. In both cases these devices attempt to spread the forces that would otherwise act on the hip and pressure sores, to the soft tissues surrounding the hip where there is not the same degree of problem with tissue viability.

Many devices in use consist of a protective pad that is held in place by a pocket in a garment, a belt and other straps. There are a number of problems with this kind of device. They are unsightly, whether worn under or over the person's everyday clothing. Such protective pads have a tendency to move with respect to the person's body, and may therefore not be in the correct position to protect the hip from injury (patient monitoring with one type of hip protector has found the device to consistently be positioned approximately 3 to 5 cm proximal and posterior to the centre of the greater trochanter whilst flexing from 0 to 90 degrees, lying down and moving about).

Another major problem with protective devices of the type described, is compliance, ie. individuals to whom the protective devices are prescribed do not wear them This may be because they are unsightly, or uncomfortable.

In WO 99/42011 there is described a two part hip protector which is fixed to the hip area. In this device, one part of the hip protector is a fastening means and is attachable to the lateral hip area (the greater trochanter), the fastening means being provided with a hole for marking and alignment The second part of the hip protector is a shell like structure attachable to the fastening means, and extending around and beyond the greater trochanter. The principle behind this invention is that in the event of a fall, energy is transmitted to the soft tissue surrounding the greater trochanter.

Whilst the hip protector described in WO 99/42011 attempts to distribute energy to soft tissue surrounding the greater trochanter, the fact that one part of the fastening means is attached to the skin over the greater trochanter means that shear forces are transmitted directly to this area of skin, which is often thin and weak.

As discussed above in connection with the known types of hip protector, it is essential that the protector is correctly located on the body. To locate the hip protector correctly requires the correct identification of the greater trochanter. In many accurate identification of the location of the greater trochanter by palpation is very difficult, even for trained medical staff. Failure to correctly identify the position of the greater trochanter may lead to the hip protector being positioned such that an edge of the protector lies on or off the greater trochanter. In such a scenario, a fall could lead to the energy being directed through, as opposed to away from, the greater trochanter. Whilst WO 99/42011 provides a bole to assist with the positioning of the hip protector, if the position of the greater trochanter cannot be identified, then the positioning aid will be of little assistance.

It would therefore be desirable to provide an improved positioning assistance means.

It is known that the anterior superior iliac crest (ASIC) is easy to identify by palpation, even in obese people.

Experimentation has found that across the population there is a constant relationship between the position of the GT and the ASIC. The distance between the GT and ASIC is approximately 11 cm (+/−1 cm), this being the substantially the same for males and females.

Observation has established that the GT moves by approximately 1.0 cm during flexion posteriorly and distally and that its area is about 3×4 cm. Thus there is a rectangular area of 4.0×5.0 cm in which the GT must be located at any time.

The invention therefore seeks to provide a device for locating the greater trochanter, and a protective device for the same.

### SUMMARY OF THE INVENTION

One aspect of the invention provides a hip protector as specified in claim 1.

Another aspect of the invention provides a device which is part of the hip protector device for protecting the greater trochanter as specified in Claim 20.

Another aspect of the invention provides a template for use in locating a region to be protected around the greater trochanter as specified in Claim 23.

Another aspect of the invention provides a template for use in locating an area in which the greater trochanter is located as specified in Claim 25.

By accurately determining the position of the region around the greater trochanter the device provides for effective protection thereof. The protector is equally useful for protection against falls and as a dressing to protect against pressure sores. By attaching the protector to the skin around the perimeter of the padded region, shear forces on the skin are reduced from the vulnerable area around the greater trochanter. Following hip surgery there is a need to monitor the heeling process of the wounds created by the surgery. The transparent nature of the padded region and the film used in attaching the protector to the leg allow the wound to be monitored without removal of the protector. One embodiment of the invention allows the protector to indicate whether the protector has been subjected to a force exceeding a certain threshold value. This is achieved by impregnating the silicone pad with frangible dye-filled capsules which fracture when subjected to a certain force.

### BRIEF DESCRIPTION OF THE DRAWINGS

In the drawings, which illustrate a preferred embodiment of the invention:

FIG. 1 is a front view of the part of the human skeleton including the left hip joint;

FIG. 2 is a side view of the part of the human skeleton including the hip joint;

FIG. 3 is a view of a template;

FIG. 4 is a side view of the template attached to a patient's leg;

FIG. 5 is a plan view of a first embodiment of a hip protector,

FIG. 6 is a plan view of a second embodiment of a hip protector,

FIG. 7 is a cross-sectional elevation of the hip protector illustrated in FIG. 5; and

FIG. 8 is a cross-sectional elevation of a hip protector including impact indication means.

### DETAILED DESCRIPTION OF THE PREFERRED EMBODIMENTS

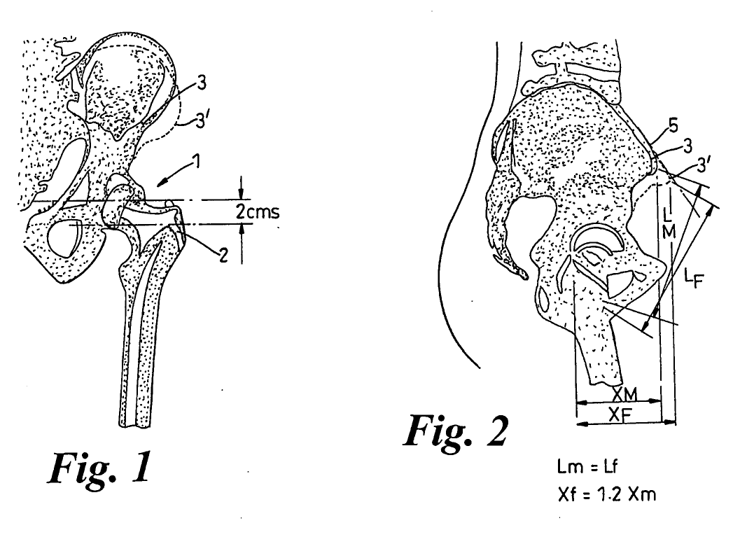

FIGS. 1 and 2 illustrate the part of the human skeleton including the hip 1. Hip protectors serve to protect the hip by dissipating energy away from the greater trochanter (GT) 1. In many individuals the GT 1 is very difficult locate by palpation. However, the anterior superior iliac crest 3 (for women and 3′ for men) is relatively easy to locate by palpation

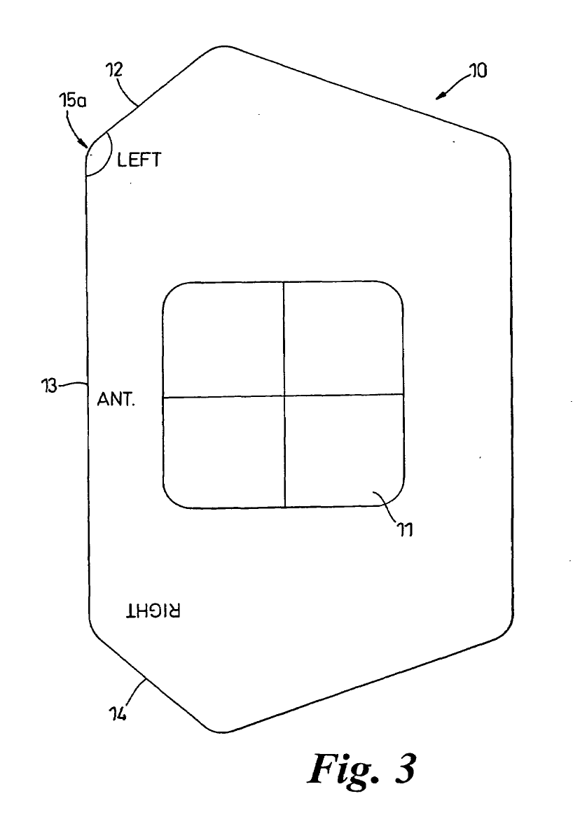

The template 10 illustrated in FIG. 3 can be used on either left or right legs and includes a central window 11, which when the template is correctly positioned defines the protected area around the GT. The window 11 is rectangular and is approximately 4.5 cm high×5.5 cm wide. The template includes short edges 12 and 14, each inclined to a long edge 13. As can be seen from broken line 5 in FIG. 2, the iliac crest is inclined to the vertical

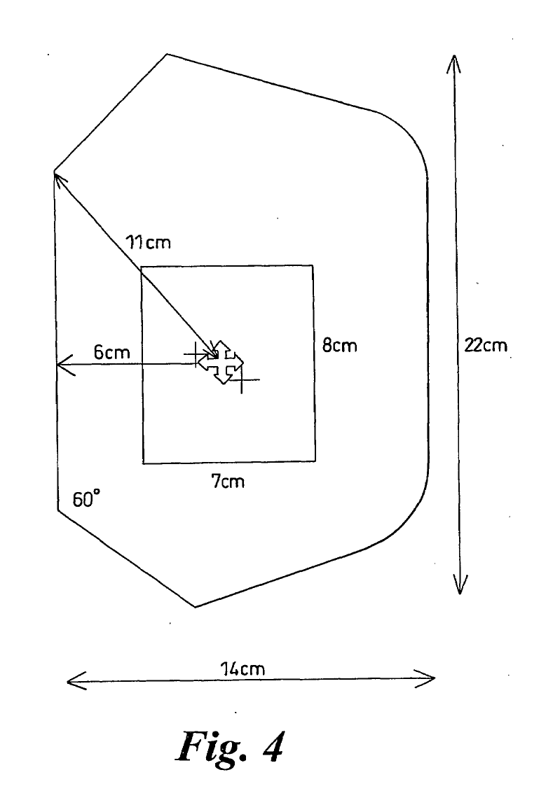

When the ASIC has been located by palpation, the template 10 is placed on the side of the leg (see FIG. 4) with the long edge 13 substantially vertical. If the template 10 is placed on the left leg, the short edge 12 is aligned parallel with the ASIC and the corner 15 overlays the anterior ridge of the ASIC. If the template 10 is placed on the right leg, the short edge 14 is aligned with the ASIC. Once the template is aligned, the protected area is the area bounded by the window 11. In this example, the template 10 is made of a clear plastics sheet

Template 10 is of use in simply locating the area around the greater trochanter. For example template 10 could be used by a surgeon to accurately locate this region prior to commencing surgery.

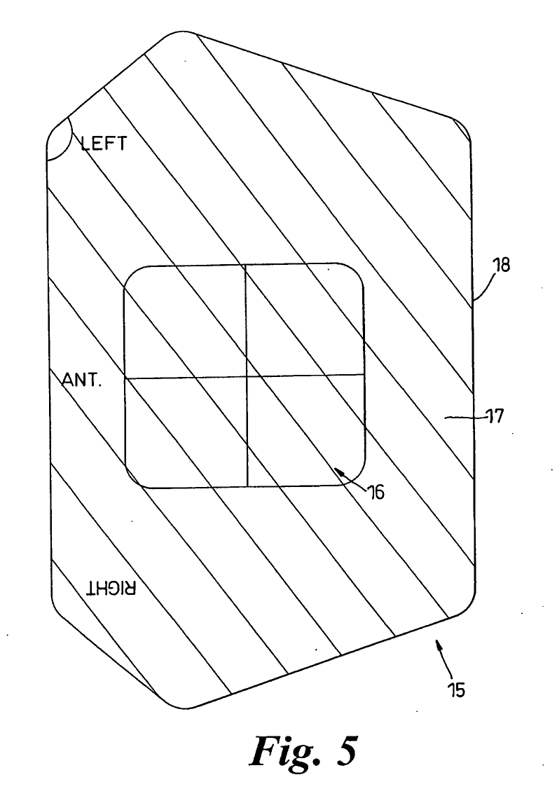

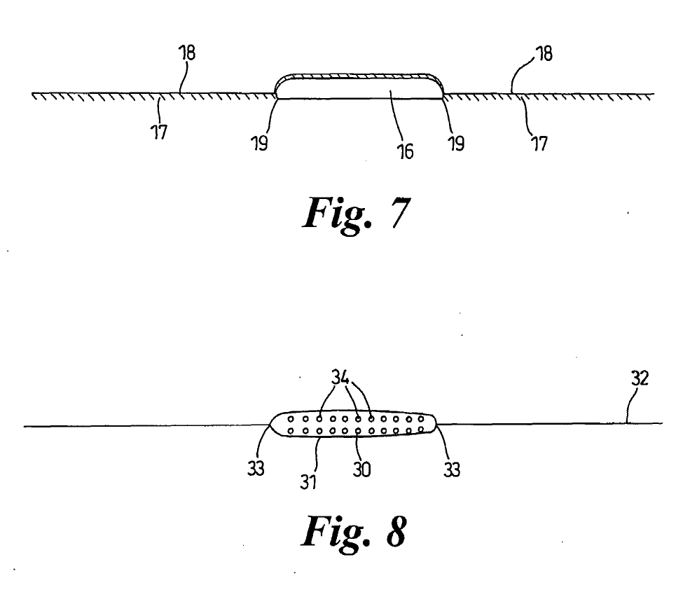

Referring now to FIGS. 5 and 7, a hip protector 15 comprises a padded region 16. In the example the padding comprises a 12 mm thick piece of hard silicone, which is mounted on an adhesive layer 17, the adhesive layer 17 being the coating of one side of a film 18 in the form of a template of the type illustrated in FIG. 3. No adhesive is applied to the surface of the silicone in facing the skin. It has been found that a 12 mm thick piece of hard silicone affords a reduction of peak force transmitted to the GT of approximately 60%.

In the example, the film forming the template and having one side coated with an adhesive layer 18 is a hydrocolloid material, which is sufficiently elastic to stretch with the skin, allows the skin to breath and prevents water entering the pad. The film 18 is also transparent, which is advantageous for medical staff applying the protector, and subsequently monitoring the patient, since the protector can be accurately positioned and the condition of the skin under the template can be observed through the protector.

A protective film (not shown) is adhered to the adhesive surface of the film 18, this film being removed prior to use.

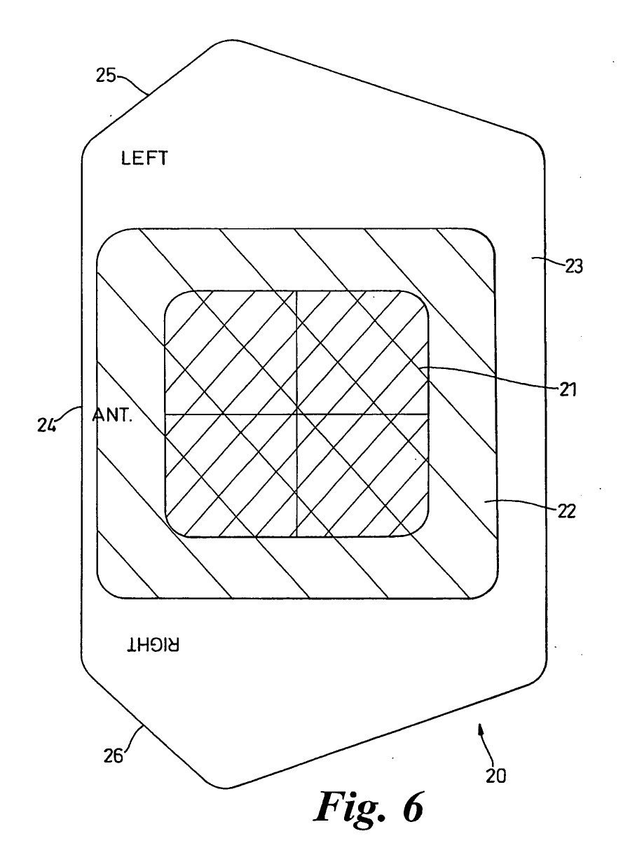

The hip protector 20 illustrated in FIG. 6 is similar to that shown in FIG. 5 except that the film 23 forming a template (as shown in FIG. 3) is disposable to leave attached to the hip a protective pad 21 attached to the leg by adhesive layer 22. The hip protector 20 comprises a padded region 21 of hard silicone mounted on an adhesive layer 22, the adhesive being covered by a plastics film 23 in the form of a template. No adhesive is applied to the surface of the padded region facing the skin. The adhesive layer is a hydrocolloid material, which is sufficiently elastic to stretch with the skin, allows the skin to breath and prevents water entering the pad.

The film 23 is transparent and is adhered to the outer surface of the adhesive layer 22 much less strongly than the under surface of the adhesive layer 22 adheres to the skin, thereby allowing the film 23 to be pulled off the said layer 22 when the protector has been applied, without removing the protector, leaving the padded region 21 and the adhesive layer 22 as the attached hip protector.

The adhesive layer 22 may also be transparent to facilitate monitoring of the region of skin under the hip protector whilst in situ.

An alternative embodiment of the hip protector is illustrated in FIG. 8. The hip protector is generally of the type illustrated in FIG. 7, with a silicone pad adhered to the underside of a hydrocolloid film 32 in the form of a template. However, the silicone pad 30 is impregnated with frangible capsules 34 containing dye of a different colour to the pad 30. The capsules are designed to fracture under a predetermined load, thereby releasing the dye into the pad 30, indicating that an impact has occurred. In the example illustrated in FIG. 8, the silicone pad may be translucent or transparent The predetermined load under which the capsules are designed to fracture corresponds to a load that could damage the hip. A range of hip protectors could be produced, the capsules in each one of the range being designed to fracture under a different load. Such a range of hip protectors would be useful since an impact which might fracture the hip of an individual having osteoporosis might be significantly lower to another individual without osteoperosis.

The edges 33 of the silicone pad 30 are rounded so as to reduce the possibility of the pad itself damaging the skin to which the protector is adhered.

1. A hip protector comprising a padded region and an adhesive region surrounding the padded region, and a template, the template comprising a first element for alignment with an anterior superior iliac crest, a second element for alignment with a substantially vertical axis, and a third element which is substantially aligned with the greater trochanter when the first and second elements are aligned respectively with the anterior superior iliac crest and the vertical axis.

2. A hip protector according to claim 1, wherein in use the padded region covers a vulnerable area around the greater trochanter.

3. A hip protector according to claim 1, wherein the material forming the padded material is transparent or translucent.

4. A hip protector according to claim 1, wherein the material forming the padded region includes silicone.

5. A hip protector according to claim 4, wherein the padded region includes hard silicone.

6. A hip protector according to claim 4, wherein the padded region includes soft silicone.

7. A hip protector according to claim 1, wherein the template includes indicia defining the vulnerable area around the greater trochanter to be protected.

8. A hip protector according to claim 1, wherein the first and second elements each consist of an edge of the template, and wherein the edges intersect.

9. A hip protector according to claim 8, wherein the third alignment element is approximately 11 cm from the point of intersection of the said edges.

10. A hip protector according to claim 8, wherein the third alignment element is approximately 6 cm from the second alignment element.

11. A hip protector according to claim 1, wherein the padded region is substantially rectangular, the rectangle being the same size and shape as the vulnerable area.

12. A hip protector according to claim 1, wherein the template includes two of said first elements, one providing for alignment with a left hand side anterior superior iliac crest, and the other providing for alignment with a right hand side anterior superior iliac crest.

13. (canceled)

14. A hip protector according to claim 1, wherein the adhesive region is formed from a breathable material having adhesive applied to one side thereof.

15. (canceled)

16. A hip protector according to claim 14, wherein the material is a hydrocolloid.

17. A hip protector according to claim 1, wherein the padded region includes impact indication means.

18. A hip protector according to claim 17, wherein the indication means comprises at least one dye containing frangible capsule.

19. (canceled)

20. (canceled)

21. (canceled)

22. A dressing in the form of a hip protector as claimed in claim 1.

23. A template for use in locating a region to be protected around the greater trochanter, the template comprising a first element for alignment with an anterior superior iliac crest, a second element for alignment with a substantially vertical axis, and a third element which is substantially aligned with the greater trochanter when the first and second elements are aligned respectively with the anterior superior iliac crest and the vertical axis.

24. (canceled)

25. A template for use in locating an area around the greater trochanter, the template comprising a first element for alignment with an anterior superior iliac crest, a second element for alignment with a substantially vertical axis, and a third element which is substantially aligned with the greater trochanter when the first and second elements are aligned respectively with the anterior superior iliac crest and the vertical axis.

26. A template according to claim 25, wherein the template is made of a transparent plastics material.
